(19) United States
(12) Patent Application Publication
(10) Pub. No.: US 2009/0304775 A1
(43) Pub. Date: Dec. 10, 2009

(54) Drug-Exuding Orthopedic Implant

(76) Inventors: Ashok V. Joshi, Salt Lake City, UT (US); Akash Akash, Salt Lake City, UT (US); John Howard Gordon, Salt Lake City, UT (US)

Correspondence Address: CERAMATEC, INC., 2425 SOUTH 900 WEST, SALT LAKE CITY, UT, 84119, US

(21) Appl. No.: 12/477,884

(22) Filed: Jun. 3, 2009

Related U.S. Application Data
(60) Provisional application No. 61/058,878, filed on Jun. 4, 2008.

(51) Int. Cl. A61K 9/00; A61K 47/30; A61P 19/00

(52) U.S. Cl. 424/426; 424/423; 514/772.3

(57) ABSTRACT

An apparatus in accordance with the present invention may include an orthopedic implant having one or more voids integrated into a surface thereof. A beneficial agent may be deposited into each void, and a regulator element may substantially cover an open end of thereof. In this manner, the regulator element may regulate delivery of the beneficial agent through the open end of the voids over a period of time.

### RELATED APPLICATIONS

This application claims priority to U.S. Provisional Patent No. 61/058,878 filed on Jun. 4, 2008 and entitled DRUG ELUTING ORTHOPEDIC IMPLANTS. This application further claims priority to U.S. patent application Ser. No. 11/744,560 filed on May 4, 2007 and entitled FULLY OR PARTIALLY BIORESORBABLE ORTHOPEDIC IMPLANT and to U.S. patent application Ser. No. 11/561,856 filed on Nov. 20, 2006 and entitled POROUS METAL AND CERAMIC IMPLANTS FOR LOAD BEARING APPLICATIONS AND DRUG DELIVERY. The above-referenced applications are hereby incorporated by reference.

### BACKGROUND OF THE INVENTION

1. Field of the Invention

This invention relates to orthopedic implants for human and animal use, and more particularly to orthopedic implants capable of exuding beneficial agents such as medicine, drugs, and the like.

2. Background

Orthopedic implants are often used to replace missing, damaged, or diseased bone or tissue. A spinal implant, for example, may be implemented to separate and cushion the vertebrae in place of a damaged or diseased intervertebral disc. Similarly, a hip implant may be implemented to replace a damaged or diseased hip joint to permit continued use and movement.

While such orthopedic implants are generally manufactured of structurally sound, biologically inert materials, the surgical process for implementing the implant may result in post-operative infection at the surgical site. In fact, recent studies have shown that the infection rate for implant-related surgeries is almost five times higher than the accepted standard for post-operative wound infection, which should be less than one percent (1%). This may result in increased costs to the patient, as well as increased rates of morbidity and mortality. Such infections may also result in increased expenditures in hospital resources, including expensive diagnostic tests and additional surgeries.

Beneficial agents such as antibiotics, bone growth factors, anti-inflammatory agents, pain killers, and other beneficial drugs may be provided to patients to enhance the healing or integration process and avoid post-operative infection. Usually, however, these agents must be injected or administered orally to the patient. It may be difficult, therefore, to maximize the effects of such agents at the surgical site, or to avoid systemic side effects. Further, the effectiveness of such agents may depend on frequent, consistent administration.

In view of the foregoing, what is needed is an apparatus that functions as an orthopedic implant while independently delivering a beneficial agent to the surgical site. Beneficially, such an apparatus would demonstrate superior biological performance and improve the healing process after surgery. Further, such an apparatus would enable modification of an existing orthopedic implant to controllably exude a beneficial agent at a desired rate over a desired period of time. Such apparatus and methods are disclosed and claimed herein.

### SUMMARY OF THE INVENTION

The invention has been developed in response to the present state of the art and, in particular, in response to the problems and needs in the art that have not yet been fully solved by currently available orthopedic implants. Accordingly, the invention has been developed to provide novel apparatus and methods for providing orthopedic implants adapted to exude a beneficial agent. The features and advantages of the invention will become more fully apparent from the following description and appended claims and their equivalents, and also any subsequent claims or amendments presented, or may be learned by practice of the invention as set forth hereinafter.

An apparatus in accordance with certain embodiments of the present invention may include an orthopedic implant having one or more voids integrated into a surface thereof. A beneficial agent may be deposited into each void, and a regulator element may substantially cover an open end thereof. The regulator element may regulate delivery of the beneficial agent through the open end over a period of time.

In some embodiments, the beneficial agent may be encapsulated in, or combined with, a polymer, such as poly lactic-co-glycolic acid (“PLGA”), which hydrolyzes when exposed to water. The regulator element may regulate access to the beneficial agent to control a rate of hydrolysis of the polymer.

In one embodiment, the regulator element may be infiltrated with the beneficial agent. In other embodiments, the regulator element may include a porosity to release the beneficial agent at a desired rate over a desired period of time. In some embodiments, the porosity may be functionally graded.

A method for modifying an existing orthopedic implant to exude a beneficial agent in accordance with certain embodiments of the present invention may include obtaining an orthopedic implant and forming at least one void in its surface. A beneficial agent may be deposited in the at least one void, and a regulator element may be applied to an open end thereof to regulate delivery of the beneficial agent through the open end over a period of time.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings in which:

FIG. 1A is a perspective view of a hip implant having voids integrated therein in accordance with embodiments of the invention;

FIG. 1B is a cross-sectional view of the implant of FIG. 1A, taken along line 120;

FIG. 2 is a perspective view of a spinal implant having a void covered by a regulator element;

FIG. 3 is a cross-sectional view of an orthopedic implant having a beneficial agent deposited into voids in accordance with certain embodiments of the invention;

FIG. 4 is a cross-sectional view of an orthopedic implant having a regulator element applied to its surface;

FIG. 5 is a cross-sectional view of an orthopedic implant having a porous plug inserted into a void thereof;

FIG. 6 is a cross-sectional view of an orthopedic implant having a void covered with a functionally graded porous plug;

FIG. 7 is a cross-sectional view of an orthopedic implant having an inwardly tapered void;

FIG. 8 is a cross-sectional view of an orthopedic implant having an outwardly tapered void;

FIG. 9 is a cross-sectional view of an orthopedic implant with a surface roughened to create irregular voids;

FIG. 10 is a cross-sectional view of an orthopedic implant with irregular voids and a regulator element applied to controllably release a beneficial agent deposited therein;

FIG. 11 is a cross-sectional view of an orthopedic implant having a porous plug infiltrated with a beneficial agent;

FIG. 12 is a cross-sectional view of a regulator element with internal channels used to cover a void of an orthopedic implant; and

FIG. 13 is a flow chart illustrating a method for modifying an orthopedic implant to exude a beneficial agent in accordance with certain embodiments of the invention.

### DETAILED DESCRIPTION OF THE INVENTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

As used herein, the term “orthopedic implant,” or simply “implant,” may refer to a medical device, including devices used to replace or supplement damaged, diseased or missing bone or tissue. Such orthopedic implants may be implemented in the spine, extremities, knees, face, hips, joints, or any other region of the body known to those in the art. For example, the orthopedic implant may be a spinal implant, a hip implant, a knee implant, a facial implant, a shoulder implant, an elbow implant, cranial implant, ankle implant a wrist implant etc.

The term “beneficial agent” refers to any agent or medicament known to provide a beneficial effect to a patient, including an antimicrobial agent, a bactericidal agent, an anti-inflammatory agent, an anti-cancer agent, a pain-relieving agent, a local drug delivery agent, a bone growth agent, or the like.

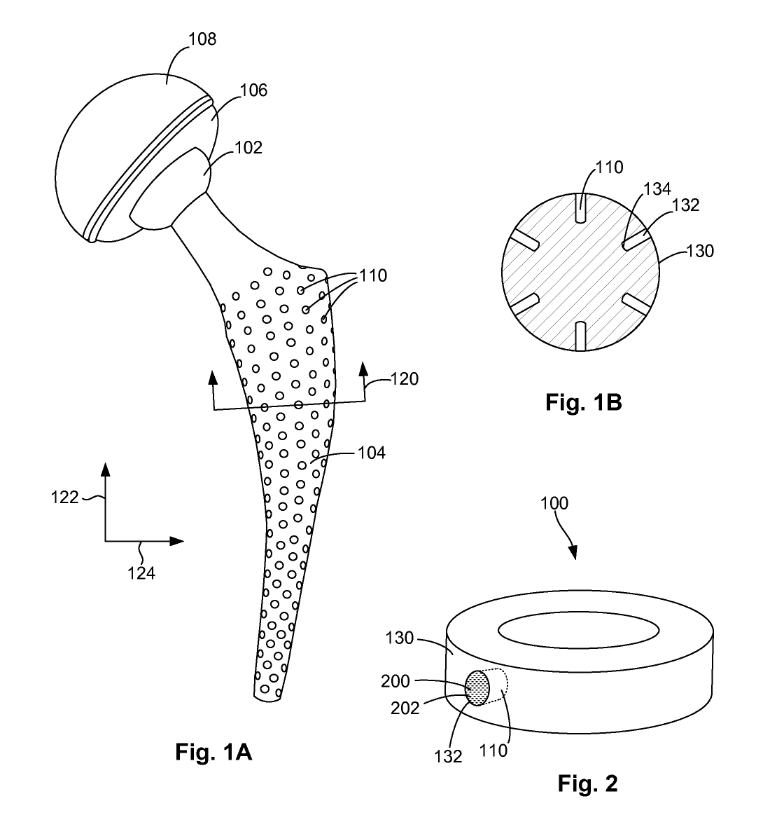

Referring now to FIG. 1, in one embodiment, an apparatus in accordance with the present invention may include an orthopedic implant 100 to replace damaged, diseased, or missing tissue in the hip. Although particular reference is made herein to a hip implant 100, the principles taught herein may be readily applied to other types of load-bearing or non-load-bearing orthopedic implants. Thus, the illustrated implant 100 is simply one of many possible embodiments of an implant 100 that may take advantage of the apparatus and methods disclosed herein.

The implant 100 may be a standard, off-the-shelf implant having a hip ball 102 connected to a stem 104. The hip ball 102 may fit into a liner 106 which may, in turn, be inserted into a metal shell 108. The metal shell 108 may be anchored to a patient's pelvis. The stem 104 may be inserted into the patient's femur. Typically, the hip ball 102 is constructed of metal or ceramic, while the liner 16 may be made of polyethylene, metal, or ceramic.

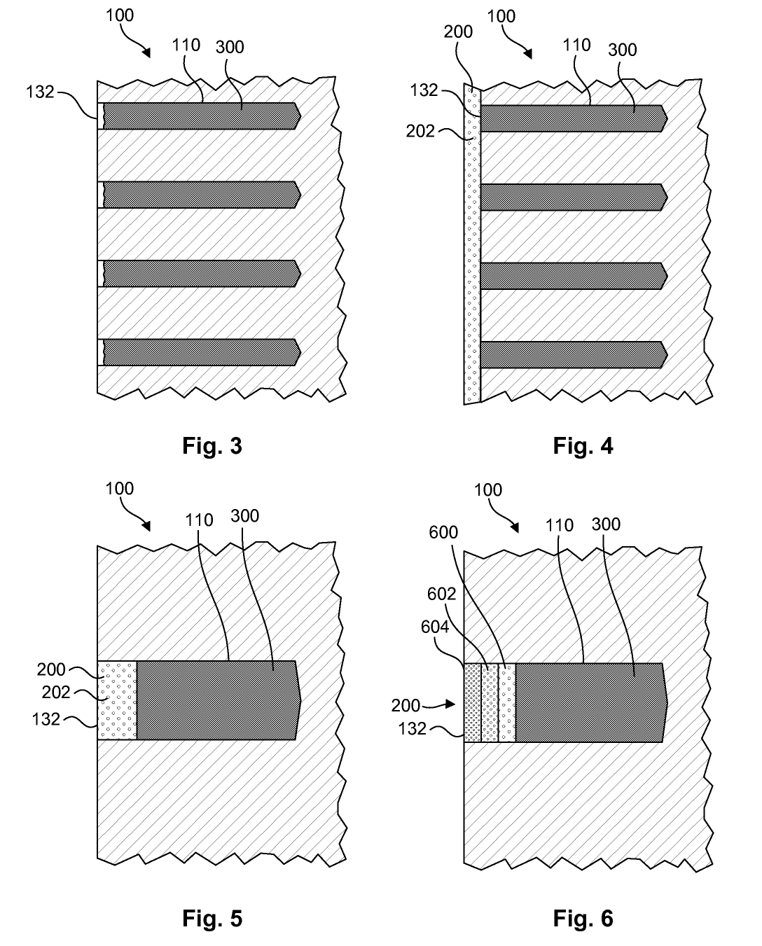

In certain embodiments, a standard, off-the-shelf implant 100 may be modified to include one or more voids 110 configured to substantially retain a beneficial agent, as discussed in more detail with reference to FIG. 3 below. Such voids 110 may be micro-drilled or otherwise integrated into an external surface 130 of the implant 102. In some embodiments, for example, a void may be integrated into a surface 130 of the implant 102 by laser machining, etching, acid bathing, drilling, mechanical or chemical means, or by any other means known to those of skill in the art.

In some embodiments, a void 110 may include, for example, a hole, a channel, a pore, a pit, a rib, a slot, a notch, a well, a groove, indentation, or the like. Each void 110 may extend partially or completely through the implant 100 in various directions, including perpendicular to its external surface 130. In certain embodiments, the voids 110 may be tapered along a length thereof, may be regular or irregular, and may have an aspect ratio ranging from 0.0000001 to 10,000,000. The voids 110 may include various cross-sectional shapes including, for example, circular, oval, rectangular, square, and irregular.

FIG. 1B is a cross-section taken along line 120 of FIG. 1A. As shown, multiple voids 110 may be integrated into a surface 130 of an implant 100 and spaced substantially equidistantally in a lateral direction 124 thereof. Likewise, in certain embodiments, voids 110 may be integrated into a surface 130 of an implant 100 and spaced substantially equidistantally in a longitudinal direction 122 thereof. In other embodiments, void 110 spacing may be substantially random.

As shown in FIG. 1B, each void 110 may include an open end 132 and a closed end 134. The open end 132 may substantially correspond to the external surface 130. As discussed in more detail below, a beneficial agent may be deposited into each void 110 such that it may exit the open end 132 at a desired rate over a desired period of time. This method of passive administration of a beneficial agent directly to the surgical site may facilitate effective drug delivery and promote patient well-being.

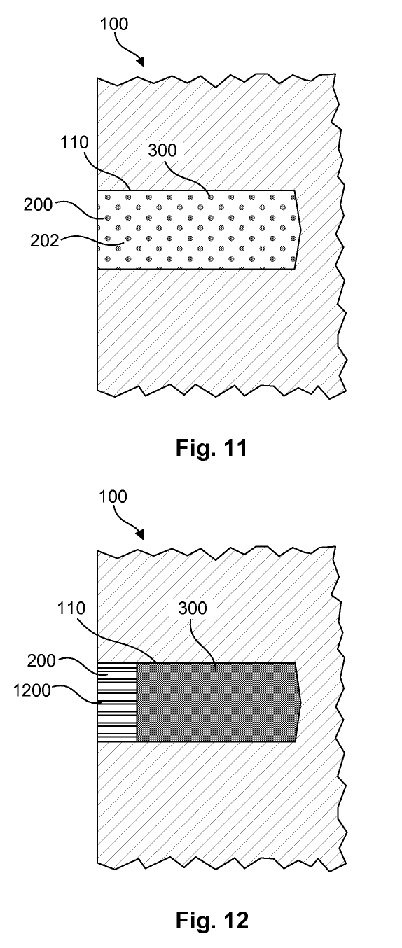

Referring now to FIG. 2, some embodiments of the present invention may include a regulator element 200 applied to the open end 132 of a void 110. The spinal implant 100 shown in FIG. 2 includes a void 110 having a regulator element 200 substantially covering an open end 132 thereof. The regulator element 200 may facilitate retention of a beneficial agent within the void 110, while permitting the beneficial agent to gradually exude from the regulator element 200 at a predetermined rate over a predetermined period of time. It will be appreciated by those of skill in the art that the reference to exude as used herein throughout includes elute, dissolve, seep, discharge, emit, or other means by which beneficial agent may come into contact with its surroundings. In some embodiments, as discussed in more detail with reference to FIG. 11 below, the regulator element 200 may be infiltrated with the beneficial agent 300. In these embodiments, the beneficial agent could be a liquid or a solid suspended in a liquid (i.e. a suspension).

A regulator element 200 in accordance with certain embodiments of the present invention may include, for example, a plug, a membrane, a film, heat, pressure, a material that affects flow or viscosity, an orifice, a conduit, a tortuous path, or any other suitable element known to those in the art. The regulator element 200 may be constructed of metal, ceramic, polymer, plastic, or the like. Further, the regulator element 200 may be bio-inert and/or bio-degradable, and may resorb, either fully or partially, into the body. As discussed in more detail below, the beneficial agent is unmixed and captured in the void 110 by a resorbable regulator 200 or regulator element 200. In this configuration, the regulator captures the beneficial agent in the void 110 until the regulator 200 resorbs, either fully or partially, allowing fluids such as body fluids to access the beneficial agent within the void 110. In this embodiment, the beneficial could be a solid, or a gel, or be viscous.

In certain embodiments, the regulator element 200 may include pores 202 ranging in size between about 10 nm and about 10 mm. These pores 202 may provide through-porosity, and form a regular recurring or random distribution pattern through the regulator element 200. The strength, rate of biodegradability and/or porosity of the regulator element 200 may be customized to control the release rate of the beneficial agent over the desired period of time.

Referring now to FIG. 3, in one embodiment, multiple voids 110 may be integrated into a surface 132 of an orthopedic implant 100. In some embodiments, each void 110 may be substantially uniform in size and shape, and may be constructed to retain a predetermined quantity of a beneficial agent 300. An open end 132 of each void 110 or the void 110 itself may be dimensioned and/or shaped to permit the beneficial agent 300 to exit the open end 132 at a predetermined rate over a predetermined period of time via capillary action or other such mechanism known to those in the art. In some embodiments, as discussed in more detail with reference to FIG. 4 below, a regulator element 200 may be applied to the open end 132 of each void 110 to regulate a flow of the beneficial agent 300 from the void 110 to an external location.

The beneficial agent 300 may include one or more chemicals or medications including, for example, cancer-related drugs, pain-related drugs, bone growth factors, anti-viral drugs, anti-inflammatory drugs, genetics-related drugs, anti-bacterial drugs, and/or any other suitable chemical or medication known to those in the art.

In certain embodiments, the regulator material may be combined with or dispersed within the beneficial agent 300. It will be appreciated by those of skill in the art that the beneficial agent 300 may be dispersed within the regulator material, such as a polymer. As used herein, if one of the regulator material or the beneficial agent is “dispersed within” the other, both dispersions are contemplated. For example, in one embodiment, the beneficial agent 300 may be combined with one or more biocompatible polymers that decompose over time when exposed to water. This depot material, consisting of the composite beneficial agent 300 and dissolvable polymer, may enable the beneficial agent 300 to be released over a predetermined period of time and at a predetermined rate, based on the molecular weight, composition, and form of the polymer.

In one embodiment, the depot material may include a beneficial agent 300 encapsulated by, or combined with, a regulator element 200 in the form of a polymer such as poly lactic-co-glycolic acid (“PGLA”), glycolic acid (“GA”), poly lactic acid (“PLA”) polyglycolic acid, poly(glycolide-co-lactide), poly(p-dioxanone), poly(glycolide-co-trimethylene carbonate), polylactic acid, polyurethane, polyalkyl 2-cyanoacrylates, polyanhydride, polyorthoesters, and the like.

Other regulator elements 200 may include materials that may or may not be absorbable, partially absorbable or bio-degradable. For example, the regulator element 200 used with the beneficial agent can include material such as Teflon, Parylene, hydrogel, polyvinylchloride, silicone, polyester, polymethyl methacrylate, polyacrylonitrile, polyamide, polyethylene terepthalates, acetal, polyethylene, polypropylene, polystyrene, Nylon, Silk, Catgut, Elastomers, Polyvinyl acetate, Cellulose Acetate Pthalate, Hydroxypropyl Cellulose, Carboxymethyl cellulose, ethyl cellulose, Methyl Cellulose, Collagen, Zein, Gelatin, Natural rubber, guar gum, gum agar, albumin, honey, and the like.

The ratio of polymers or other regulator elements 200 in the depot material and the molecular weights of such regulator elements 200 may affect the rate of decomposition of the polymer in the body. In certain embodiments, for example, the polymer may break down into lactic acid and glycolic acid, which may be readily absorbed by and eliminated from the body. As the polymer decomposes, the beneficial agent 300 may be released. The time frame for this release may range from a few days to several months. The composition of body-friendly polymers and beneficial agents may be adjusted to achieve various delivery rates.

Indeed, a depot material in accordance with embodiments of the invention may be specially formulated to release the active beneficial agent 300 in a consistent way over a prolonged period of time. One advantage of such a long-acting depot material may include increased compliance with medication requirements due to a reduction in the frequency of dosing, as well as more consistent serum concentrations. The depot material or beneficial agent may be solid, oil-based, a liquid, gel, suspension, or may take any other form known to those in the art. In certain embodiments, the depot material or medication may include certain forms of a beneficial agent 300, such as decanoate salts or esters.

Referring now to FIG. 4, a regulator element 200 may be applied to the open end 132 of each void 110 to regulate a flow of the beneficial agent 300 from the void 110 to an external location. In some embodiments, the regulator element 200 may be applied by dipping, rolling, brushing, spraying, inserting, injecting, sintering, plasma spraying, pulsed laser deposition, chemical vapor deposition, or adhering the regulator element 200 to the open end 132 of each void 110, or by any other means known to those in the art.

As shown in FIG. 4, in some embodiments, the regulator element 200 may include a membrane, film, or other layer applied substantially indiscriminately to a surface 130 of the implant 100. In this manner, the regulator element 200 may effectively cover the open end 132 of each void 110, while also forming a substantially continuous layer over the surface 130 of the implant 100. In other embodiments, the regulator element 200 may be particularly applied to substantially cover only the open end 132 of each void 110.

Referring now to FIG. 5, in some embodiments, the regulator element 200 may include a substantially porous plug or cap inserted into or attached to the open end 132 of each void 110. Such a plug or cap may be constructed of metal, ceramic, polymer, plastic, or any other suitable material known to those in the art. The plug or cap may be bio-inert, partially or fully resorbable, and/or biodegradable.

Further, as mentioned above, the plug or cap may include pores 202 ranging in size between about 10 nm and about 10 mm. The strength, rate of biodegradability and/or porosity of plug may control the period of time and rate at which the beneficial agent is released.

In certain embodiments, the porous plug may regulate discharge of the beneficial agent 300 by restricting flow of the beneficial agent 300 out of the void 110, as well as by controlling access to the beneficial agent 300 by water or other fluid around the implant 100. In this manner, the porous plug or other regulator element 200 may control a rate of hydrolysis or breakdown of a polymer encapsulating the beneficial agent 300 or combined with the beneficial agent 300, thereby controlling a release rate of the beneficial agent 300. It will be appreciated by those of skill in the art that combining may include dispersion of the beneficial agent within the polymer or vice versa, either evenly or unevenly.

In certain embodiments, the beneficial agent 300 may be deposited in each void 110 by immersing the implant 100 in the beneficial agent 300, injecting the beneficial agent 300 into the void 110 via a syringe or pipette, using vacuum infiltration, or by any other means known to those in the art. The porous plug or other regulator element 200 may then be inserted into each void 110, or otherwise applied to an open end 132 of each void 110, to substantially retain the beneficial agent 300 within the void 110 and to regulate a flow of the beneficial agent 300 therefrom. In some cases, external machining may be performed to create a smooth surface across the plug or other regulator element 200 that is substantially flush with the main body of the implant 100. In other cases, external machining may be performed to roughen or otherwise texture the surface of the regulator element 200 and/or implant 100 to facilitate bone ingrowth.

Referring now to FIG. 6, in certain embodiments, the regulator element 200 may include a functionally graded porosity to enable controlled discharge of the beneficial agent 300. For example, as shown in FIG. 6, the regulator element 200 may include a first layer 600 having a first porosity, a second layer 602 having a second porosity, and a third layer 604 having a third porosity. The first layer 600, by itself, may include a structure and porosity to enable a substantially fast flow, while the second and third layers 602 and 604, by themselves, may include structures and porosities to enable successively slower flow rates. The layers 600, 602 and 604 may be combined as desired to achieve a particular flow rate that is different from the flow rate that could be achieved by any individual layer 600,602 or 604.

In certain embodiments, the thickness, porosity, and strength of the layers may be adjusted to produce a regulator element 200 with desired properties. For example, in certain embodiments the thickness of the layers 600, 602, 604 may be varied to adjust the flow rate through the regulator element 200. In other embodiments, stronger layers may be placed adjacent to weaker layers to provide strength and structural integrity thereto. In other embodiments, the different layers 600, 602, 604 may have different rates of resorption or degradation as a way to control the delivery rate of beneficial agent from the void. Thus, different variables may be adjusted to provide a regulator element 200 with desired strength, dimensions, resorption rate, and delivery rate. Furthermore, the regulator element 200 may have different phases with different characteristics to control and customize the delivery or access of beneficial agent to the surrounding environment. In other embodiments, instead of internal porosity, the regulator element 200 may include internal channels that are functionally graded to enable controlled discharge of the beneficial agent 300. The cross-sectional size or diameter of such internal channels may range from about 10 nm to about 10 mm.

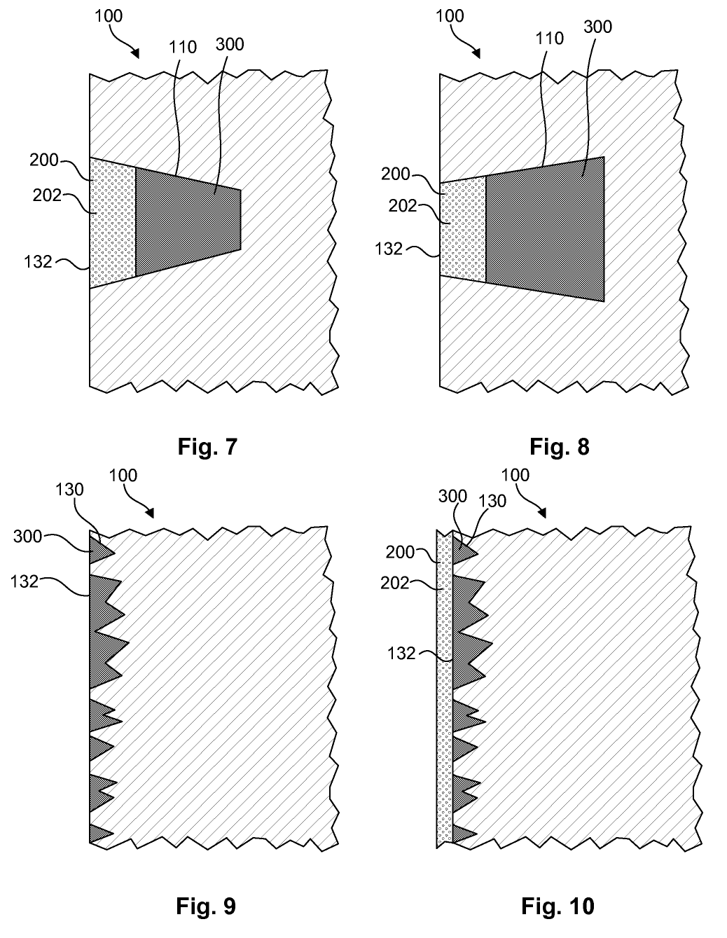

Referring now to FIGS. 7 and 8, voids 110 integrated into an implant 100 in accordance with embodiments of the present invention may be sized and shaped as desired to substantially retain a beneficial agent 300. For example, as shown in FIG. 7, a void 110 may be tapered inwardly to facilitate retention of a porous plug or other regulator element 200 covering the beneficial agent 300. In other embodiments, as shown in FIG. 8, a void 110 may be tapered outwardly to retain a greater volume of beneficial agent 300.

Referring now to FIG. 9, some embodiments of the invention may include an implant 100 having a roughened, textured, or irregular surface 130 adapted to retain a beneficial agent 300. In some embodiments, regular or irregular voids 110 may be formed in the surface 130 of an existing implant 100 by roughening, texturing, abrasion, machining, etching, acid bathing, drilling, mechanical or chemical means or by any other means known to those in the art.

A beneficial agent 300 may then be deposited in the voids 110 for later release. For example, the implant 100 may be immersed in a pool of the beneficial agent 300, the beneficial agent 300 may be injected into the voids 110 via a syringe or pipette, or vacuum infiltration may be used. In other embodiments, the beneficial agent 300 may be rolled, brushed, or sprayed into the voids 110, or deposited into the voids 110 by any other means known to those in the art.

In certain embodiments, a regulator element 200 may be applied to substantially cover an open end 132 of such voids 110. As shown in FIG. 10, for example, a regulator element 200 may be dipped, rolled, brushed, sprayed, inserted, injected, adhered, or otherwise applied to the open end 132 of the voids 110. As in other embodiments, the regulator element 200 may include pores 202, internal channels, or the like to regulate discharge of the beneficial agent 300.

Referring now to FIG. 11, in some embodiments, the regulator element 200 may be infiltrated with a beneficial agent 300 using vacuum infiltration, for example. In other embodiments, the regulator element 200 may be infiltrated with the beneficial agent 300 by any method known to those in the art.

In some embodiments, the beneficial agent 300 may be encapsulated in a microsphere or combined with a polymer that breaks down when exposed to water or other fluid. This combination of the beneficial agent 300 and polymer may infiltrate pores 202 in the regulator element 200 such that the beneficial agent 300 is gradually released from the pores 202 as more of the polymer is exposed to water or other fluid over time.

Referring now to FIG. 12, certain embodiments of a regulator element 200 may include internal channels 1200 or conduits, instead of pores, to regulate delivery of the beneficial agent 300. The internal channels 1200 may be through-channels having a size and shape to permit release of the beneficial agent 300 at a desired rate and over a desired period of time.

In some embodiments, the internal channels 1200 may provide a tortuous path such that the length, size, shape, and/or direction of the path may be selectively adjusted to achieve a desired flow rate. In other embodiments, the regulator element 200 may include a combination of various layers, where each layer incorporates internal channels 1200 having certain densities, sizes, and shapes. The thickness, number, and structure of each layer may provide a regulator element 200 having a desired strength, dimensions, and delivery rate.

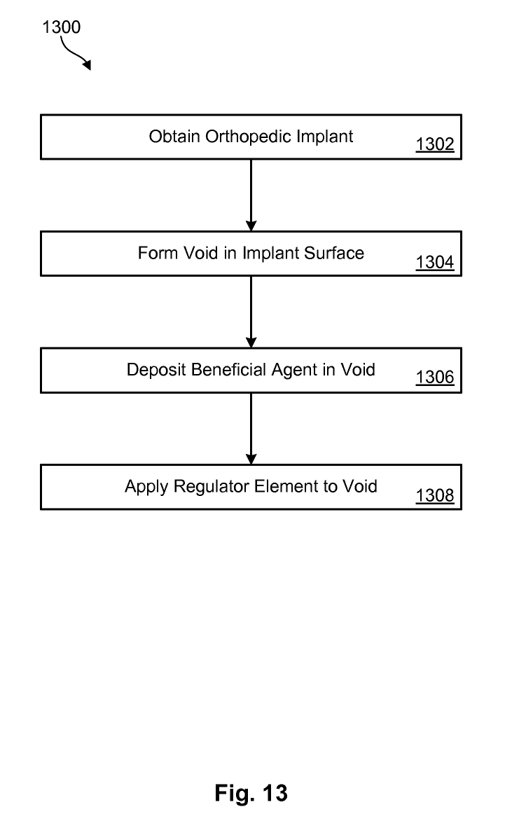

Referring now to FIG. 13, a method 1300 in accordance with embodiments of the invention may include obtaining 1302 an orthopedic implant such as a spinal implant, a hip implant, a knee implant, a facial implant, a shoulder implant, an elbow implant, a wrist implant, or the like. One or more voids may be formed 1304 in a surface of the implant. These voids may be formed as part of a retrofitting process, or during manufacture of the orthopedic implant. A void may include, for example, a hole, a channel, a pore, a pit, a rib, a slot, a notch, a well, indentation, or a groove.

A beneficial agent may then be deposited 1306 in the voids. In some embodiments, the beneficial agent may be encapsulated or combined with a polymer that hydrolyzes when exposed to water. A regulator element may be applied 1308 to an open end of the voids to regulate delivery of the beneficial agent through the open end over a period of time. In certain embodiments, as discussed in more detail above, the regulator element may be infiltrated with the beneficial agent.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. An apparatus comprising:
an orthopedic implant;
at least one void integrated into a surface of the orthopedic implant;
a beneficial agent deposited in the void; and
a regulator element substantially covering an open end of the at least one void to regulate delivery of the beneficial agent through the open end over a period of time.

2. The apparatus of claim 1, wherein the beneficial agent comprises a form selected from a solid, a liquid, a suspension, a gel, and combinations thereof.

3. The apparatus of claim 1, wherein the regulator element comprises at least one of a metal, a ceramic, a polymer, and a plastic.

4. The apparatus of claim 3, wherein the beneficial agent is combined with a polymer that hydrolyzes when exposed to water.

5. The apparatus of claim 3, wherein the beneficial agent is encapsulated by a polymer.

6. The apparatus of claim 3, wherein the beneficial agent is dispersed within a polymer.

7. The apparatus of claim 3, wherein the polymer comprises one of poly lactic-co-glycolic acid (“PGLA”), glycolic acid (“GA”), poly lactic acid (“PLA”) poly(glycolide-co-lactide), poly(p-dioxanone), poly(glycolide-co-trimethylene carbonate), polylactic acid, polyurethane, polyalkyl 2-cyanoacrylates, polyanhydride, polyorthoesters and combinations thereof.

8. The apparatus of claim 7, wherein the regulator element further regulates access to the beneficial agent by fluid to control a rate of hydrolysis of the polymer.

9. The apparatus of claim 1, wherein the regulator element comprises a porosity to release the beneficial agent at a predetermined rate over a predetermined period of time.

10. The apparatus of claim 1, wherein the regulator element is selected from the group consisting of a layer, a plug, and a cap.

11. The apparatus of claim 1, wherein the regulator element is infiltrated with the beneficial agent.

12. The apparatus of claim 1, wherein the regulator element is fully or partially resorbable.

13. An apparatus comprising:
an orthopedic implant;
at least one void integrated into a surface of the orthopedic implant;
a beneficial agent deposited in the void, said beneficial agent comprising a form chosen from a liquid and a suspension; and
a porous plug inserted into an open end of the at least one void to regulate delivery of the beneficial agent through the open end over a period of time.

14. The apparatus of claim 13, wherein the porous plug is resorbable.

15. An apparatus comprising:
an orthopedic implant;
at least one void integrated into a surface of the orthopedic implant;
a beneficial agent deposited in the void, said beneficial agent comprising a form chosen from a solid and a gel; and
a resorbable porous plug inserted into an open end of the at least one void to regulate delivery of the beneficial agent through the open end over a period of time.

16. An apparatus comprising:
an orthopedic implant;
at least one void integrated into a surface of the orthopedic implant;
a beneficial agent deposited in the void;
a regulator element comprising a polymer chosen from poly lactic-co-glycolic acid (“PGLA”), glycolic acid (“GA”), poly lactic acid (“PLA”) poly(glycolide-co-lactide), poly(p-dioxanone), poly(glycolide-co-trimethylene carbonate), polylactic acid, polyurethane, polyalkyl 2-cyanoacrylates, polyanhydride, polyorthoesters and combinations thereof;
wherein the beneficial agent is combined with the polymer that hydrolyzes when exposed to water.

17. A method for modifying an existing orthopedic implant to exude a beneficial agent, the method comprising:
obtaining an orthopedic implant;
forming at least one void in a surface of the orthopedic implant;
depositing a beneficial agent in the at least one void;
applying a regulator element to an open end of the at least one void to regulate delivery of the beneficial agent through the open end over a period of time.

18. The method of claim 17, wherein the orthopedic implant is selected from the group consisting of a spinal implant, a hip implant, a knee implant, a facial implant, a shoulder implant, an elbow implant, and a wrist implant.

19. The method of claim 17, wherein the at least one void is selected from the group consisting of a hole, a channel, a pore, a pit, a rib, a slot, a notch, a well, and a groove.

20. The method of claim 17, further comprising at least one of encapsulating and combining the beneficial agent with a polymer that hydrolyzes when exposed to water.

21. The method of claim 17, wherein applying the regulator element comprises at least one of dipping, rolling, brushing, spraying, inserting, injecting, and adhering the regulator element to the open end of the at least one void.

22. The method of claim 17, further comprising infiltrating the regulator element with the beneficial agent.

23. The method of claim 17, wherein depositing the beneficial agent in the at least one void comprises at least one of immersing the orthopedic implant into the beneficial agent, injecting the beneficial agent into the at least one void, and infiltrating the beneficial agent into the at least one void via vacuum infiltration.

24. The method of claim 23, wherein the regulator element comprises a porosity to release the beneficial agent at a predetermined rate over a predetermined period of time.

25. The method of claim 24, wherein the porosity is functionally graded.
